(19) United States
(12) Patent Application Publication
(10) Pub. No.: US 2025/0114223 A1
(43) Pub. Date: Apr. 10, 2025

(54) Aortic Stent Graft Device

(71) Applicant: Daniel McGlade, Narraweena (AU)

(72) Inventor: Daniel McGlade, Narraweena (AU)

(21) Appl. No.: 18/909,872

(22) Filed: Oct. 8, 2024

Related U.S. Application Data
(60) Provisional application No. 63/543,234, filed on Oct. 9, 2023.
(60) Provisional application No. 63/544,598, filed on Oct. 17, 2023.

(51) Int. Cl. A61F 2/95; A61F 2/94

(52) U.S. Cl. CPC A61F 2/95; A61F 2/94

(57) ABSTRACT

A stent delivery system for deployment of a stent along the inner surface of an outer curve of an aortic lumen. The system includes a stent graft, a retraction chord, and an introducer with a channel for conveying the chord along an interior surface of the introducer, where the stent is deployed to at least partially cover the origin of the branching vessel off the main aortic lumen.

### FIELD OF THE INVENTION

The present disclosure relates to improvements in systems, devices, and methods designed to reline a vascular lumen to exclude from the circulation a diseased segment and the positioning and accurate placement of such devices in a subject.

### BACKGROUND OF THE INVENTION

Conventional stents inserted into the aortic passage typically involve two main problems. A first problem is accurate positioning and deployment of the proximal (leading) end of the stent, and final position of the proximal end. A final proximal end position that is too far forward (inserted) runs the high risk of covering a critical artery such as the carotid artery, leading to potentially dire medical consequences. At the same time, not placing the proximal end far enough forward runs the risk that the stent will not develop an adequate seal, leaving the diseased segment of the artery exposed to the heamodynamics of the circulation, again with potentially dire consequences. Therefore, proper final placement of the proximal end of a stent is critical.

Another problem typically encountered is obtaining a sufficient length of graft to healthy vessel wall apposition (the seal zone) around the proximal end of the stent to inhibit blood flow between the stent and the vessel wall. A problem typically encountered at the inner curve of the (lower, near-side) aortic arch is a lack of vessel wall apposition of the leading edge of the stent and commonly called “bird beaking”. Conventional device design has focused heavily on preventing bird beaking. However, stent placement must also focus on maximising the length of the seal zone, with the most common limiting factor being the vessels on the outer curve (far-side) of the aortic arch. Accordingly, there exists a need to provide an improved stent device which provides more accurate proximal end placement on the outer curve of the aortic arch to minimise risk of adversely affecting blood flow in crucial anatomical structures whilst maximising the seal zone.

### SUMMARY

The present disclosure in one preferred aspect provides for stent delivery system configured to accurately deploy a stent graft for placement along the inner surface of an outer curve of an aortic lumen. The system includes a stent graft including a proximal end for placement first into the aortic lumen, and a distal end opposite the proximal end, a fabric graft membrane, and a frame configured to expand the membrane. The system includes a stent graft with an interior retraction chord/fiber guide located along an interior of a wall of the stent that is configured to contact the interior surface of the outer curve of the aortic lumen once the stent graft is finally positioned. The system further includes a retraction chord/fiber configured to extend along the retraction chord/fiber guide of the stent graft prior to deployment of the stent graft; and an introducer catheter configured to exteriorly introduce the stent graft along a guide wire into the aortic lumen for deployment of the stent graft, the catheter including a leading, proximal end, a length, and an interior channel configured for conveying and movement of the retraction chord therethrough along at least a portion of the length of the catheter. The catheter includes a retraction chord outlet distal to the leading end, and a retraction chord inlet spaced apart and distal from the retraction chord outlet.

In another preferred aspect, there is provided a stent delivery system configured to accurately deploy a stent graft for placement in along the inner surface of an outer curve of an aortic lumen. The system includes a stent graft including a proximal end, a distal end opposite the proximal end, and a length from the proximal end to the distal end, a fabric graft membrane having an exterior surface, a frame structure configured to expand the membrane, and a retraction chord/fiber guide located primarily but not exclusively along the exterior surface of the graft membrane. The system further includes a retraction chord; and an introducer catheter configured to exteriorly introduce the stent graft along a guide wire into the aortic lumen for deployment of the stent graft. The catheter includes a leading, proximal end, a length, and an interior passage configured for conveying and movement of the retraction chord/fiber therethrough along at least a portion of the length of the catheter. The catheter includes a retraction chord/fiber outlet distal to the leading end, and a retraction chord/fiber inlet spaced apart and distal from the retraction chord/fiber outlet, the retraction chord/fiber inlet being spaced from the leading end of the catheter a distance greater than the length of the sent graft.

In further preferred aspect, there is provided a method for inserting and positioning a stent graft within the aortic lumen. The method includes inserting a guidewire into the aortic lumen; inserting a catheter along the guidewire, the catheter having a stent graft along an exterior surface of the catheter, the catheter being inserted so that the stent graft partially covers the origin of a branching vessel off the main aortic lumen; deploying the stent graft while the graft at least partially covers the origin of the branching vessel; retracting the proximal end of the stent graft contacting the inner surface of the outer curve of the aortic lumen in short increments with a retraction chord/fiber to withdraw the proximal end of the stent graft while the stent graft is in a deployed state to uncover the origin of the branch vessel.

The branching vessel may be a left subclavian artery, left carotid artery, vertebral artery, or barchio cephalic trunk. The retraction chord is preferably located exteriorly of the stent graft once deployed. The retraction chord may be located interiorly of the stent graft once deployed. Alternatively, the retraction chord may be located both interiorly and exteriorly of the stent graft once deployed.

It is to be understood that both the foregoing general description and the following detailed description are exemplary and explanatory only and are not restrictive of the invention, as claimed. In the present specification and claims, the word “comprising” and its derivatives including “comprises” and “comprise” include each of the stated integers, but does not exclude the inclusion of one or more further integers. It will be appreciated that reference herein to “preferred” or “preferably” is intended as exemplary only.

The claims as filed and attached with this specification are hereby incorporated by reference into the text of the present description. The disclosures of U.S. Provisional Patent Application Nos. 63/543,234 and 63/544,593 are hereby incorporated by reference herein. The accompanying drawings, which are incorporated in and constitute a part of this specification, illustrate several embodiments of the invention and together with the description, serve to explain the principles of the invention.

### BRIEF DESCRIPTION OF THE FIGURES

FIG. 1 is a partial side cross sectional view of a stent delivery system for placing a stent graft in an aortic lumen with a delivery catheter in accordance with a preferred embodiment of the present disclosure, with the delivery catheter having spaced apart delivery chord apertures configured for a retraction chord travel path extending interiorly along the stent graft.

FIG. 2 is a partial expanded view of a proximal end of the stent delivery system of FIG. 1.

FIG. 3 is a partial side view of the delivery catheter of FIG. 1.

FIG. 4 is a partial perspective view of a stent delivery system for placing a stent graft in an aortic lumen with a delivery catheter in accordance with another preferred embodiment of the present disclosure, with the delivery catheter having a delivery chord aperture configured for a retraction chord travel path extending exteriorly along the stent graft.

FIG. 5 is a partial cross sectional side view of the delivery system of FIG. 4 with an exteriorly and interiorly positioned retraction chord/fiber.

FIG. 6 is a flow chart of a preferred method for placing a stent graft into an aortic passage.

FIG. 7A is a side view of the delivery system of FIG. 1 being positioned to insert the stent graft into an aortic lumen in accordance with a preferred method of use.

FIG. 7B is another side view of the delivery system of FIG. 1 being positioned to insert the stent graft into an aortic lumen, with the stent being deployed (expanded) and a portion of the stent graft covering a branch vessel from the main aortic lumen.

FIG. 8A is a side view of the delivery system of FIG. 7A being retracted distally to uncover the branch vessel.

FIG. 8B is a side view of the deployed stent graft of FIG. 8A being left in a final position after detachment of a retraction chord, and withdrawal of the guide wire and delivery catheter from the lumen.

FIG. 9 is an expanded view of the positioning of the stent graft relative to the left subclavian and left carotid artery.

FIG. 10 is an expanded view of the positioning of the stent graft to retract the leading end of the graft into the optimal seal zone distally of the left subclavian artery.

FIG. 11 is another partial perspective view of the stent delivery system of FIG. 4, with an external retraction chord travel path.

FIG. 12 is a partial perspective view of a stent delivery system for placing a stent graft in an aortic lumen with a delivery catheter in accordance with another preferred embodiment of the present disclosure, with the delivery catheter having a single retraction chord aperture and an arrangement for facilitating a retraction chord travel path interiorly along the stent.

FIG. 13 is a partial perspective view of a stent delivery system for placing a stent graft in an aortic lumen with a delivery catheter in accordance with another preferred embodiment of the present disclosure, with the delivery catheter having a single retraction chord aperture and an arrangement for facilitating a retraction chord travel path extending interiorly along the stent.

FIG. 14 is another partial perspective view of the stent delivery system of FIG. 1, with the delivery catheter having spaced apart retraction chord apertures configured for a retraction chord travel path extending interiorly along the stent graft.

FIG. 15 is a partial perspective view of a stent delivery system for placing a stent graft in an aortic lumen with a delivery catheter in accordance with another preferred embodiment of the present disclosure, with the delivery catheter having spaced apart retraction chord apertures configured for a retraction chord travel path extending exteriorly and interiorly along the stent graft.

### DETAILED DESCRIPTION OF THE DRAWINGS

Reference will now be made in detail to the present preferred embodiments of the disclosure, examples of which are illustrated in the accompanying drawings.

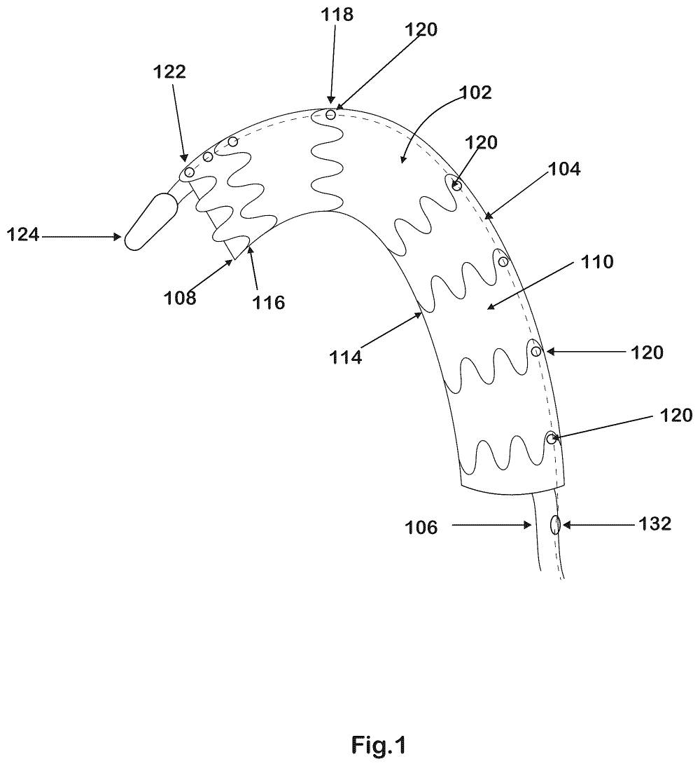

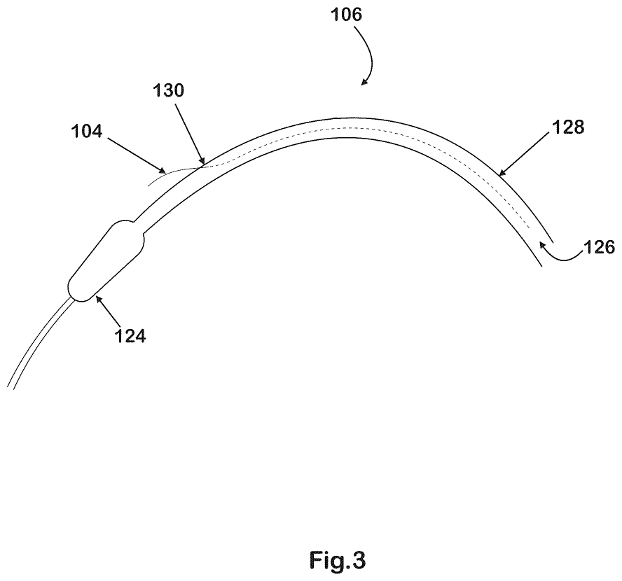

FIGS. 1 to 3 show a preferred embodiment of a stent delivery system 100 including a stent graft 102, an internal retraction chord 104, and a delivery catheter 106. In use, a guidewire 10 is inserted into a patient, and delivery catheter 106 introduced along the guidewire into the patient. Using fluoroscopy, the catheter is advanced into the aortic arch to a position covering a portion of at least one branch vessel of the aorta (e.g., left subclavian artery or left carotid artery). Following the complete deployment of the stent the surgeon then uses retraction chord 104 and digital subtraction angiography to retract (withdraw) the proximal (leading end) of the graft to a point where it no longer impedes safe blood flow through the branch vessel off the main aortic lumen. Retraction chord 104 is attached in a region of the leading end proximate an inner surface of the outer curve of the aortic arch. The preferred elements of delivery system 100 and their interrelationship are described below.

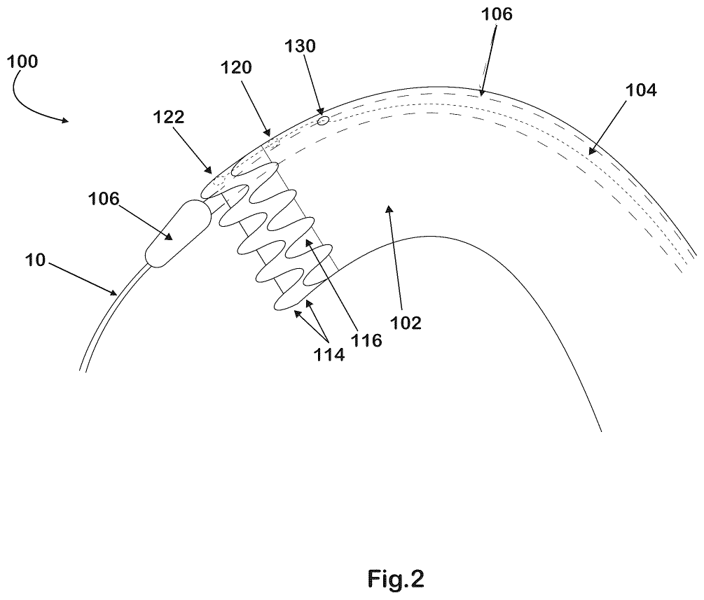

Referring now to FIGS. 1 to 3, stent delivery system 100 includes stent graft 102, retraction chord 104, and delivery catheter 106. Referring to FIGS. 1 and 2, stent graft 102 includes a leading, proximal end 108, a distal end opposite proximal end 108, and central longitudinal axis along a length from the proximal end to the distal end. Graft 102 includes a graft membrane 110 extending circumferentially around its central longitudinal axis. Graft 102 includes an expansion frame 112 integrated with the membrane. Expansion frame 112 preferably includes a plurality of expandable rings 114, preferably formed form a shape memory material or metal such as Nitinol. Rings 114 are preferably positioned to separately and independently expand. Proximal end 102 is preferably strengthened to impart an element of rigidity and or increased radial force to facilitate sealing of the proximal end 102 against the inner surface of the aorta. One exemplary way to strengthen proximal end 102 is to include multiple layers of membrane material and closely spaced rings at a leading edge region 116, and/or using a tape-like material extending from the leading edge to a point rearwardly of the leading edge sufficient to facilitate the sealing of the graft.

As shown in FIGS. 1 and 2, graft 102 includes a main internal blood flow passage through which blood will flow once graft 102 is properly positioned. The inner wall of the blood flow passage includes a retraction chord guide 118 integrated within the wall. In a preferred form, retraction guide may form a single, unitary structure, or a plurality of component parts. For example, retraction chord guide 118 may include a plurality of loops, rings or attachments 120 configured for passage of retraction chord 104 therethrough. Loops 120 may be made from the same material as membrane 110 if desired. Each ring or attachment is preferably anchored on a portion of the frame.

Graft 102 further preferably includes a retraction chord detachment point 122 internally and distal to the leading edge of proximal end 108. Chord detachment point 122 facilitates disassociation of retraction chord 104 from the graft once graft 102 is finally positioned, and the surgeon begins to withdraw other instruments from the patient. Preferably, detachment point 122 is approximately 5 mm from the proximal end of the stent, and may be configured for fixedly attaching the chord or fibre to facilitate retraction of the proximal end of the stent in contact with the outer curvature of the aortic lumen once introduced or deployed.

Retraction chord 104 is preferably made from a fibrous material and length suitable for intended purposes in cardiovascular procedures.

Referring now to FIGS. 1 to 3, delivery catheter 106 includes a leading end 124, an internal channel 126, and a trailing end with a distally located deployment handle (not illustrated). Channel 126 has a circumferentially surrounding wall 128 along a central longitudinal axis of the catheter. Wall 128 includes a retraction chord outlet 130, preferably in the region of leading end 124. Wall 128 includes a retraction chord inlet 132 distally of outlet 130 along the central longitudinal axis of catheter 106. In use, chord 104 is moveably, internally positioned to extend through a majority of the length of the catheter, and exit from catheter at outlet 130, where chord 104 is configured to run forward through to detachment point 122 and then be reversed to be oriented rearwardly away from the leading end of the graft, through internal loops 120 of graft 102. Extending through loops 120, chord 104 re-enters channel 126 through inlet 132. Chord 104 is preferably preconfigured to extend through catheter 106 and graft 102 prior to insertion and deployment, and then operated to perform retraction manipulations once graft 102 is deployed.

Graft 102 described above may have direct attachment of the retraction chord 104 to a detachable point 122 at the proximal end 108 of stent 102, without the chord being orientated reward. For example, the chord may exit channel 104 through an outlet 132 in the delivery catheter 106 either distal to the graft 102 as seen in FIG. 1, or closer to the proximal end 130 as seen in FIG. 2.

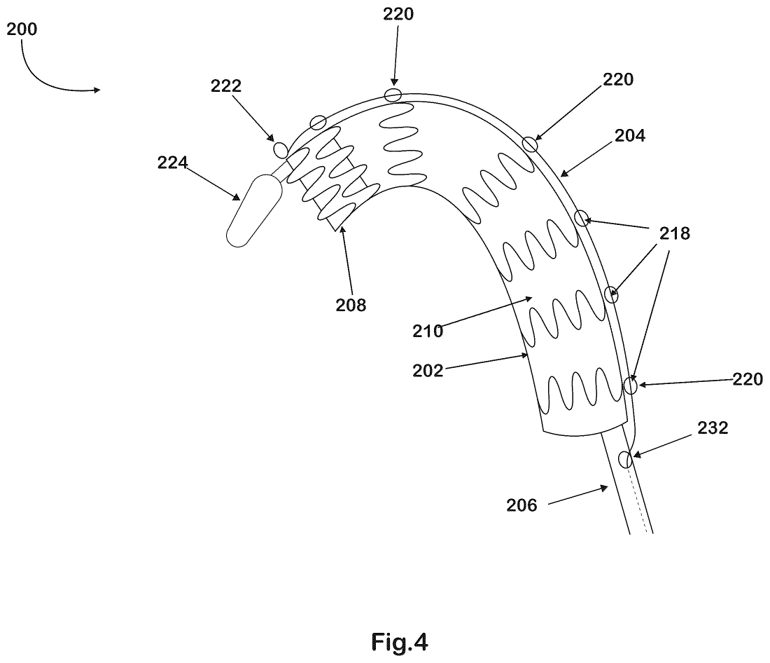

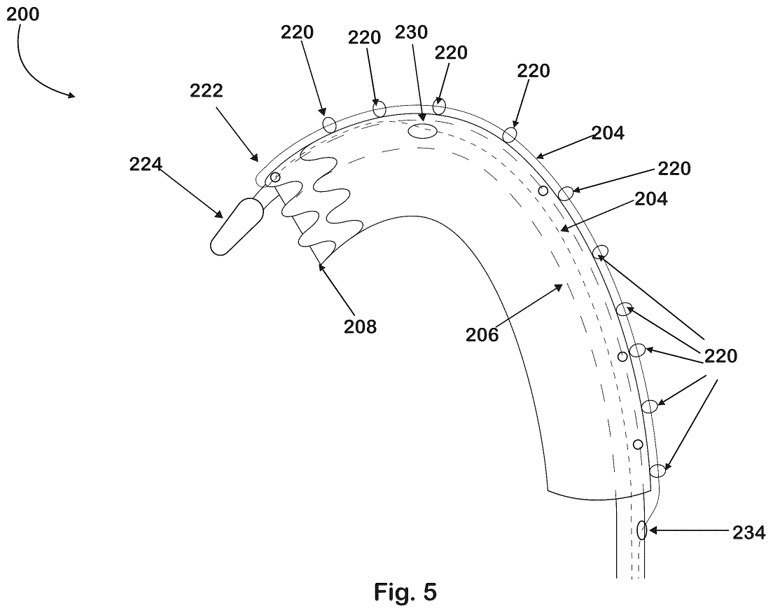

Referring now to FIGS. 4 and 5, a stent delivery system 200 is shown in accordance with another preferred embodiment of the present disclosure. The structure of system 200 is similar to that of system 100, so unless otherwise noted, the description of system 100 will be understood to apply to system 200 as appropriate. System 200 includes a stent graft 202, retraction chord 204, and delivery catheter 206. Stent graft 202 is similar to graft 102 except that graft 202 includes a retraction chord guide 218 on an exterior surface of graft membrane 210. For example, loops 220 are arrayed along the exterior surface of graft 202, preferably along the surface intended to contact the inner surface of the outer curve of the aortic lumen. To facilitate the external nature of this embodiment, retraction chord outlet 232 is positioned closer to the distal (deployment handle not shown) end of catheter 206 compared to catheter 106. Such an arrangement permits chord 204 to extend around the distal edge of graft 202 before extending proximally along the exterior surface of graft 202 through loops 220. Chord 204 extends to a point 222 at the distal end of graft, and then reverses to be oriented rearwardly away from the leading end of the graft, through internal loops to re-enter the channel of the delivery catheter 204 nearer the leading end 208 through an inlet 230, as shown in FIG. 5.

The embodiment of FIGS. 4 and 5 show a configuration which permits finite adjustments from an exterior surface orientation, particularly in the region of the inner surface of the outer curve of the main aortic lumen.

Graft 202 described above may have direct attachment of the retraction chord 204 to a detachable point 222 at proximal end 208 of the stent 206, as seen in FIG. 4. without the chord being orientated reward as seen in FIG. 5.

Preferably, the retraction chord or fibre guide is configured to remain affixed to the membrane after withdraw of any instrument use to insert the stent. Preferably, the retraction chord/fiber guide and retraction chord/fiber act to retract only a portion of membrane and or frame along the length of the stent. The proximal end of the stent includes an attachment mechanism for fixedly attaching the chord/fiber to facilitate retraction of the proximal end that contacts the outer curvature of the aortic lumen once introduced or deployed.

In the exemplary embodiments set forth above, it may be observed that multiple designs (e.g., five above) are essentially based on the same or similar two principles utilizing a retraction chord or guide.

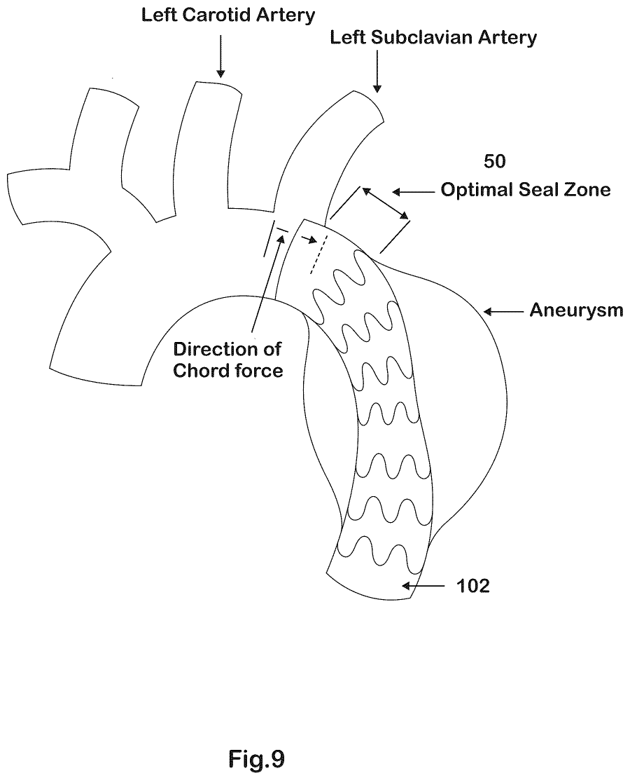

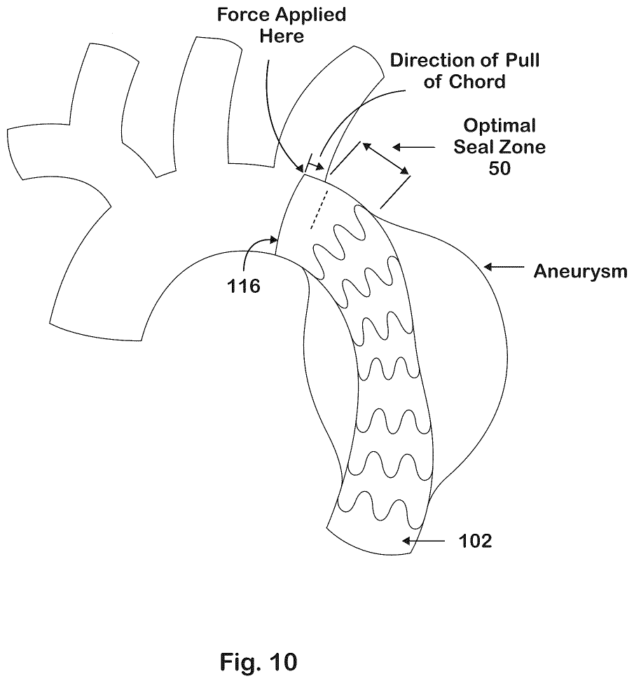

Having described preferred components of a stent deliver system, a preferred method of use 300 will now be described with reference to FIGS. 1 to 3, and 6 to 10. In use, in step 302, the surgeon advances guidewire 10 into the ascending aorta with the aid of fluoroscopy. In step 304, the surgeon inserts catheter 106 along guidewire 10 to a point preferably forward of the intended final position within the ascending aorta, e.g., forward of the left subclavian artery, or even to a point partially covering the origin of the left carotid artery. Positioning will be facilitated with radiographic imaging, or other known imaging methods. Surgeons do not ordinarily intentionally move a graft to cover or obfuscate a branch vessel of the aorta for clear detrimental consequences. However, the configuration of the delivery system in a preferred embodiment permits an intentional overlapping of the juncture of the branching passage at an initial stage of the procedure since the surgeon will partially withdraw the graft so as to permit a sufficient blood flow through the formally inhibited branch vessel

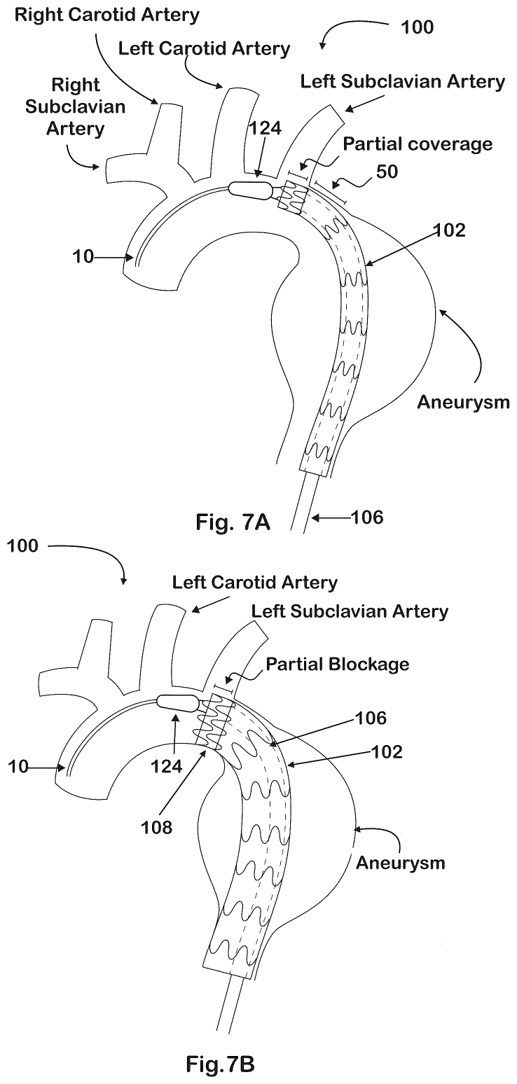

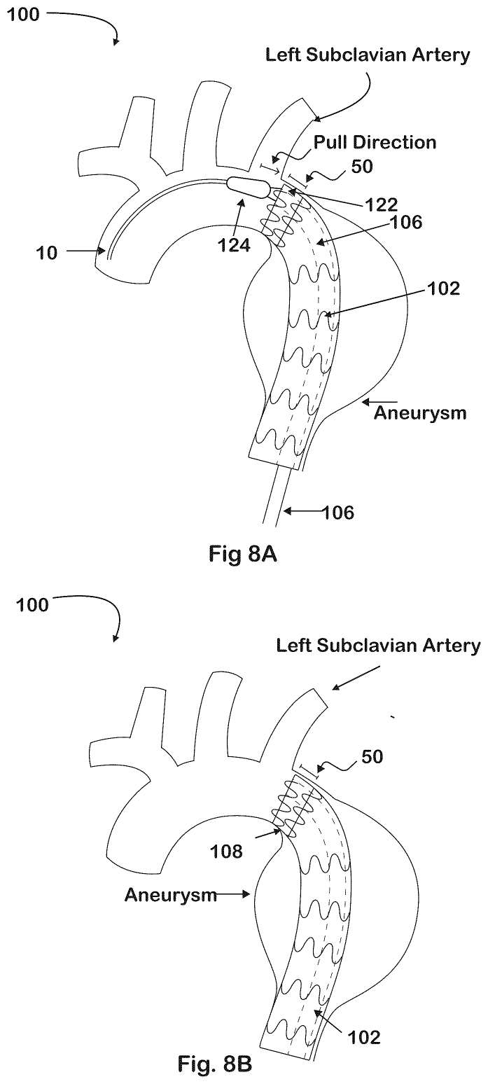

It is important to create a good seal around the leading edge of the proximal end of the graft to divert blood flow through the stent, and minimise risk of blood leaking between the stent and the inner aortic wall and potential progressive aneurysmal dilation of the aorta. The design of the delivery system of the present embodiment has a focus on contact of the proximal end of graft 102 with the inner surface of the outer curve of the aortic arch, and utilisation of the entire optimal sealing zone 50 (FIGS. 7A and 8A). In a preferred embodiment, sealing zone 50 is located rearwardly (opposite to direction of insertion) of the junction of the main aortic lumen and the left subclavian artery however may be proximal to this. This focus permits greater attention on placement of the graft relative to important branch vessels and maximising the contact of the graft with healthy aortic wall over any sealing of the graft along the inner surface of the inner curve of the aortic lumen.

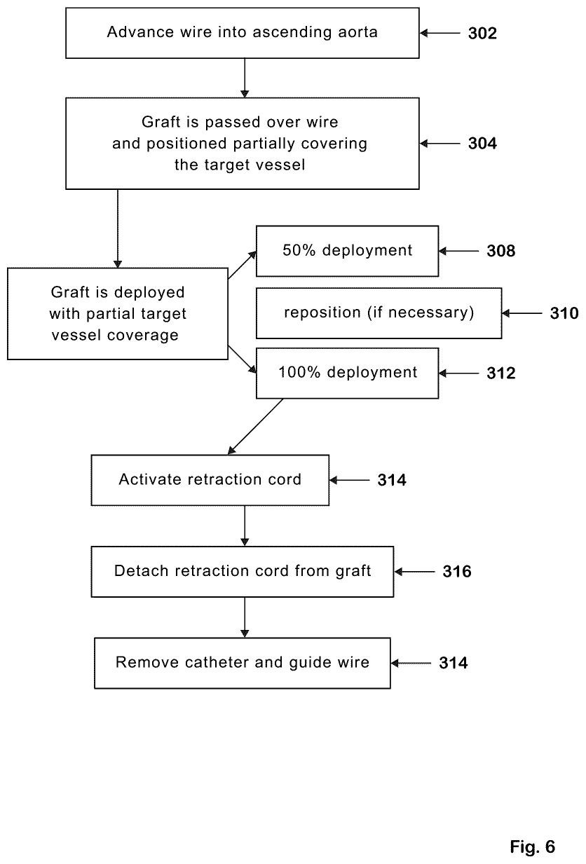

Continuing with reference to FIG. 6, once optimally positioned by the surgeon, graft 102 is deployed in step 306, preferably in stages. In a first stage 308, the graft is approximately 50% deployed (expanded), then the surgeon checks to see if any re-positioning is needed in step 310. If the positioning of the graft appears to be adequate, the surgeon then deploys the graft to 100% deployment (full expansion) in step 312.

Once fully deployed, the surgeon activates retraction chord 104 in step 314 to move at least the outer curve leading edge of proximal end 108 of graft 102 rearwardly, back along the outer curve of the aortic lumen so that the target branch vessel is not obfuscated, or causing detrimental blood flow blockage to critical anatomical regions.

Once the stent is positioned to the satisfaction of the attending surgeon, the surgeon detaches retraction chord 104 in step 316. Catheter 106 and guidewire 10 are removed in the usual way in step 318.

It will be appreciated that the steps described above may be performed in a different order, varied, or certain steps omitted entirely without departing from the scope of the present disclosure. For example, graft deployment (expansion) can be conducted in a single step rather than through a variable deployment. Where used, variable deployment can be accomplished with different amounts, and not strictly 50% as a first step, as would be appreciated by those in the field. If desired, a drug delivery step may be included (where the situation is suitable for such a step).

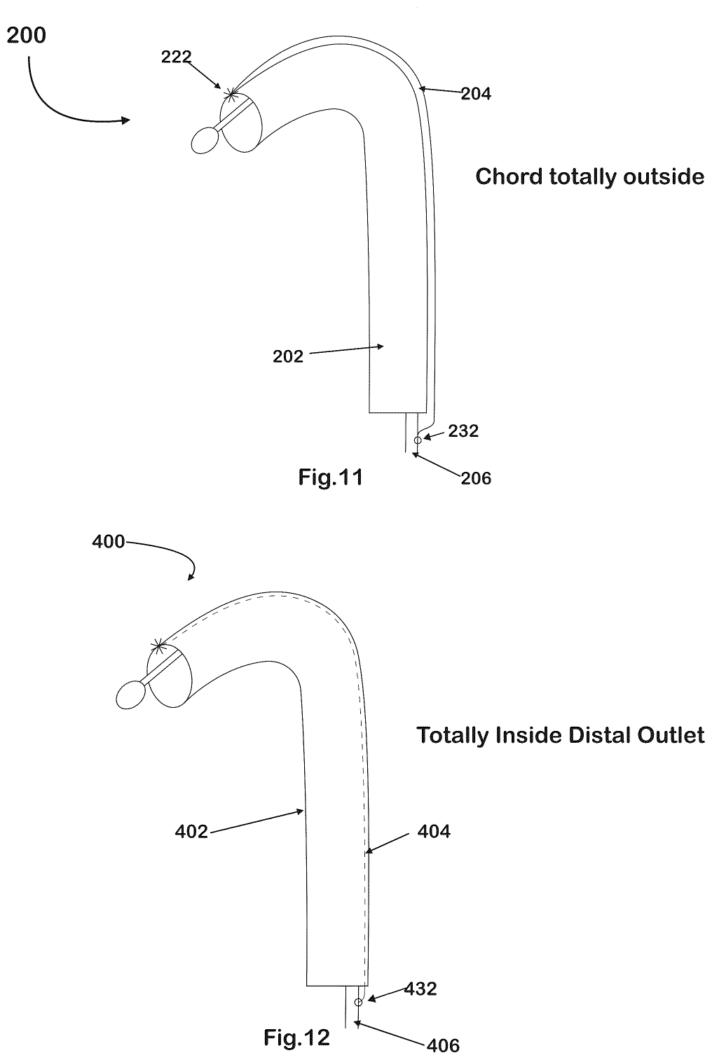

The placement of the retraction chord relative to the delivery cannula and stent graft may be achieved in several configurations to promote greater positional control and maximise seal length. For example only, FIGS. 11 to 15 show at least five configurations or arrangements that are possible for interactions between the delivery catheter, stent graft, and retraction chord. It will be appreciated that other configurations are possible as well.

FIG. 11 shows system 200 above (see, FIG. 4), where delivery catheter 206 has a single retraction chord aperture 232 that is uncovered by stent graft 202 when graft 202 and catheter 206 are engaged, and graft 202 includes an external retraction chord guide 218 configured to facilitate a retraction chord travel path extending exteriorly along stent graft 202 through loops 220 (FIG. 4), from aperture 232, to near the leading end of graft 202 where it is attached by a mechanism that will allow for detachment of the chord once the final position has been reached. The embodiment shown in FIGS. 4 and 11 is an example of a single retraction chord aperture (uncovered by the stent graft with an undeployed system) for use with an external retraction chord travel path along the graft.

Referring now to FIG. 12, a stent delivery system 400 is shown in accordance with another preferred embodiment of the present disclosure. The structure of system 400 is similar to that of system 100, so unless otherwise noted, the description of system 100 will be understood to apply to system 400 as appropriate. For system 400, graft 402 includes an internal retraction chord guide configured to facilitate a retraction chord travel path extending interiorly along stent graft 402 through internal loops, from aperture 432, to near the leading end of graft 402, where it is attached with a mechanism that can be released once the graft is in its final position. The interior loops of the graft may be configured similar to internal loops 120 shown in FIG. 1. The embodiment shown in FIG. 12 shows an example of a single retraction chord aperture (uncovered by the stent graft with an undeployed system) for use with an internal retraction chord travel path along the graft.

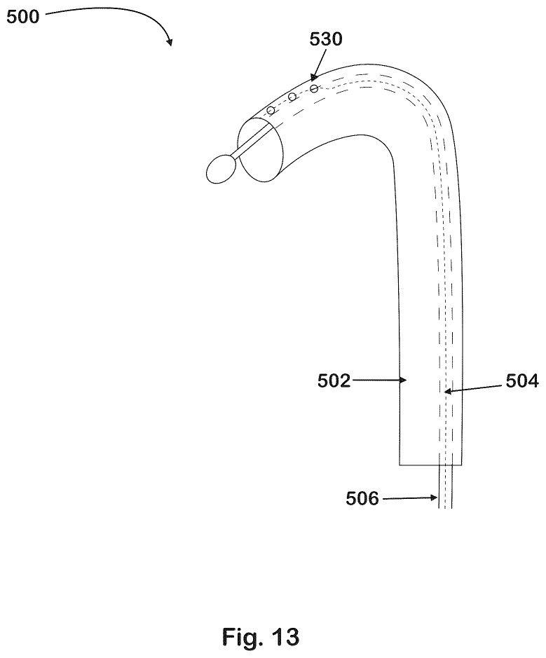

Referring now to FIG. 13, a stent delivery system 500 is shown in accordance with another preferred embodiment of the present disclosure. The structure of system 500 is similar to that of system 100, so unless otherwise noted, the description of system 100 will be understood to apply to system 500 as appropriate. For system 500, graft 502 includes an internal retraction chord guide configured to facilitate a retraction chord travel path extending interiorly through a channel in delivery catheter 506 before exiting from aperture 530 near the leading end of graft 502. The chord passes for a short distance along the internal surface of graft 502 through a plurality of guides, and then the chord is attached near the leading edge of the graft with a mechanism that allows it to be released once the graft is in the final position. The interior loops of the graft may be configured similar to internal loops 120 shown in FIG. 1. The embodiment shown in FIG. 13 shows an example of a single retraction chord aperture (covered by the stent graft with an undeployed system) for use with an internal retraction chord travel path along the graft.

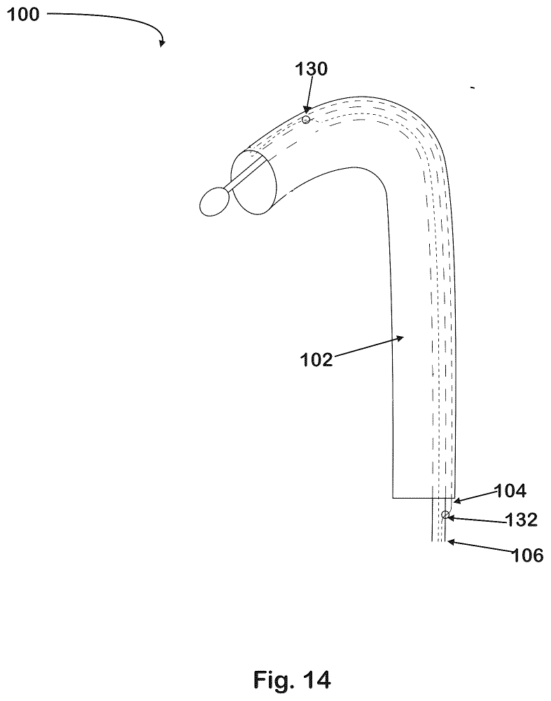

FIG. 14 shows system 100 above (FIG. 1), where delivery catheter 106 has a double retraction chord aperture configuration. The first aperture 130 (exit aperture) is located proximate the leading end of delivery catheter 106, and the second aperture 132 (re-entry aperture) is located in a spaced-apart relationship longitudinally and distally from aperture 130. Aperture 132 is uncovered by stent graft 102 pre graft deployment. Graft 102 includes an internal retraction chord guide 118 configured to facilitate a retraction chord travel path extending interiorly along stent graft 102 from exit aperture 130 to the leading edge where it reverses and travels internally through loops 120 (FIG. 1), to retraction chord aperture 132. The embodiment shown in FIGS. 1 and 14 show an example of a longitudinally-spaced double retraction chord aperture arrangement (one of the apertures being covered by the stent graft with an undeployed system) for use with an internal retraction chord travel path along the graft.

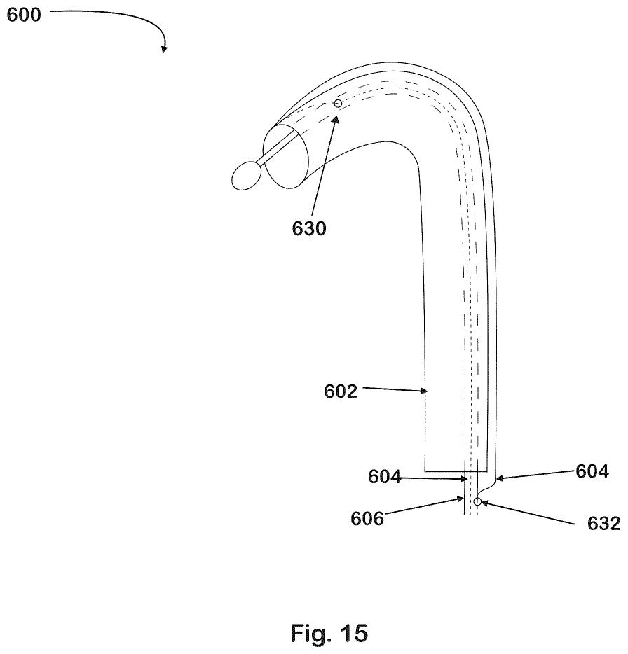

Referring now to FIG. 15, a stent delivery system 600 is shown in accordance with another preferred embodiment of the present disclosure. The structure of system 600 is similar to that of system 200, so unless otherwise noted, the description of system 200 will be understood to apply to system 600 as appropriate. For system 600, graft 602 includes an external retraction chord guide configured to facilitate a retraction chord travel path extending exteriorly along stent graft 602 through external loops, from aperture 632 to the leading end of graft 602, the chord then reverses around the leading edge and travels along the interior surface of graft 602, and to retraction chord aperture 630, where it enters and travels within a channel in the delivery catheter 606. The exterior loops of the graft may be configured similar to exterior loops 220 shown in FIG. 4. The embodiment shown in FIG. 15 shows an example of a longitudinally-spaced double retraction chord aperture arrangement (one of the apertures being covered by the stent graft with an undeployed system) for use with an external retraction chord travel path along the graft.

References to “inner” and “outer” and are for illustrative convenience only as would be appreciated by a person skilled in the art.

The foregoing description is by way of example only, and may be varied considerably without departing from the scope of the present disclosure. For example only, the expansion frame may be configured in a variety of ways as would be appreciated by those in the field. Where rings are used as part of the expansion frame, the rings may be independently moveable/expandable, or may be interconnected to one another through a structure netting. The rings may be independently moveable, yet still interconnected with a structure beyond just being integrated with the membrane fabric. For example, the retraction chord guide may act to interconnect components of the frame to one another with the base of each loop 120 extending to an expansion ring 114, and the loops being connected to one another via chord 104.

Preferably, the delivery system is configured for primary use in the aortic arch. However, one or more components may be modified for placement and use elsewhere within the patient as desired, particularly where navigation of anatomical bends and curves are present, such as in the abdominal cavity. However, for purposes of the preferred example described above, the stent has only a single blood flow passage or lumen for conveying blood flow once fully deployed, and is not bifurcated at one end as would be expected in a stent for use in the abdominal cavity.

The stent may be configured with a side passage branching from the main passage. For example, this would facilitate a second smaller stent to be placed into the side passage from one of the aortic arch branch vessels. Such a configuration may lend itself to an anatomical area such as the left subclavian or left carotid arteries to allow for perfusion of these vessels without the need for a surgical bypass. It may also facilitate the stent being placed as far proximal in the aortic arch as the brachio-cephalic trunk.

The features described with respect to one embodiment may be applied to other embodiments, or combined with or interchanged with the features of other embodiments, as appropriate, without departing from the scope of the present disclosure.

The present disclosure in a preferred form provides the advantages of greater positional control and placement compared to systems which emphasise wall apposition with respect to the inner curve of the aortic passage. Emphasising positional placement to maximise seal length over inner curve wall apposition reduces risk of catastrophic incidences (e.g., inadvertent blockage of the carotid artery). A positional focus results in better patient outcomes in terms of patient morbidity and survival of the procedure, which is inherently risky.

Other embodiments of the disclosure will be apparent to those skilled in the art from consideration of the specification and practice of forms of the embodiments disclosed herein. It is intended that the specification and examples be considered as exemplary only, with a true scope and spirit of the disclosure being indicated by the following claims.

What is claimed is:

1. A stent delivery system configured for accurate deployment of a stent graft for placement in the inner surface of an outer curve of an aortic lumen, comprising:
a stent graft including a proximal end for placement first into the aortic lumen, and a distal end opposite said proximal end, a fabric graft membrane, and a frame configured to expand said membrane, said stent graft including an interior retraction chord/fiber guide located along an interior of a wall of said stent that is configured to contact the interior surface of the outer curvature of the aortic lumen once the stent graft is finally positioned;
a retraction chord/fiber configured to extend along the retraction chord guide of said stent graft prior to deployment of said stent graft; and
an introducer catheter configured to exteriorly introduce said stent graft along a guide wire into the aortic lumen for deployment of said stent graft, said catheter including a leading, proximal end, a length, and an interior channel configured for conveying and movement of said retraction chord therethrough along at least a portion of the length of said catheter, said catheter including a retraction chord/fiber outlet distal to said leading end, and a retraction chord inlet spaced apart and distal from said retraction chord outlet.

2. The stent delivery system of claim 1, wherein said frame comprises a metal.

3. The stent delivery system of claim 1, wherein said frame comprises nitinol.

4. The stent delivery system of claim 1, wherein said retraction chord/fiber guide is located only on the interior membrane wall facing the inner surface of the outer curve of the aortic lumen.

5. The stent delivery system of claim 1, wherein said retraction chord attaches approximately 5 mm from the proximal end of the stent.

6. The stent delivery system of claim 1, wherein said retraction chord/fiber guide includes a plurality of rings or attachments configured for passage of the retraction chord therethrough.

7. The stent delivery system of claim 1, wherein said proximal end of the stent graft includes an attachment mechanism for fixedly attaching said chord/fiber to facilitate retraction of said proximal end of stent in contact with the outer curvature of the aortic lumen once introduced or deployed.

8. The stent delivery system of claim 1, wherein said retraction chord/fiber guide is configured to remain affixed to the membrane after withdraw of any instrument used to insert the stent.

9. The stent delivery system of claim 1, wherein said retraction chord/fiber guide and retraction chord/fiber act to retract only a portion of membrane and or frame along the length of the stent.

10. The stent delivery system of claim 1, wherein the stent only has a single blood flow lumen when fully deployed in a patient.

11. A stent delivery system configured for accurate deployment of a stent graft for placement in along an inner surface of an outer curve of an aortic lumen, comprising:
a stent graft including a proximal end, a distal end opposite said proximal end, and a length from said proximal end to said distal end, a fabric graft membrane having an exterior surface, a frame structure configured to expand said membrane, and a retraction chord/fiber guide located primarily along the exterior surface of said graft membrane;
a retraction chord/fiber; and
an introducer catheter configured to exteriorly introduce said stent graft along a guide wire into the aortic lumen for deployment of said stent graft, said catheter including a leading, proximal end, a length, and an interior channel configured for conveying and movement of said retraction chord/fiber therethrough along at least a portion of the length of said catheter, said catheter including a retraction chord/fiber outlet distal to said leading end, and a retraction chord/fiber inlet spaced apart and distal from said retraction chord outlet, said retraction chord inlet being spaced from said leading end of said catheter a distance greater than the length of said sent graft.

12. The stent delivery system of claim 11, wherein said frame comprises a metal.

13. The stent delivery system of claim 11, wherein said frame comprises nitinol.

14. The stent delivery system of claim 11, wherein said retraction chord/fiber guide is located only on the membrane surface facing the inner surface of the outer curve of the aortic lumen.

15. The stent delivery system of claim 11, wherein said retraction chord/fiber attaches approximately 5 mm from the distal end of the stent.

16. The stent delivery system of claim 11, wherein said retraction chord/fiber guide includes a plurality of rings or attachments configured for passage of the retraction chord/fiber therethrough.

17. The stent delivery system of claim 16, wherein each ring or attachment is anchored on a portion of said frame.

18. The stent delivery system of claim 11, wherein said proximal end of the stent includes an attachment mechanism for fixedly attaching said chord/fiber to facilitate retraction of said proximal end that contacts the outer curvature of the aortic lumen once introduced or deployed.

19. The stent delivery system of claim 11, wherein said retraction chord guide is configured to remain affixed to the membrane after withdraw of any instrument used to insert the stent.

20. The stent delivery system of claim 11, wherein said retraction chord guide and retraction chord act to retract only a portion of membrane and/or frame along the length of the stent.
